SEQUENCE LISTING (37 sequences)

SEQ ID NO: 1 - protein, 24 aa, Artificial Sequence
  Description of Artificial Sequence Synthetic peptide
  CD8a transmembrane domain
IYIWAPLAGTCGVLLLSLVITLYC

SEQ ID NO: 2 - protein, 112 aa, Artificial Sequence
  Description of Artificial Sequence Synthetic polypeptide
  CD3z intracellular domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 42 aa, Artificial Sequence
  Description of Artificial Sequence Synthetic polypeptide
  4-IBB intracellular domain
KRGRKKLLYIFKQPFMRPVQTTQEEDGCSCRFPEEEEGGCEL

SEQ ID NO: 4 - protein, 19 aa, Artificial Sequence
  Description of Artificial Sequence Synthetic peptide
  CD8a signal domain
MAWVWTLLFLMAAAQSIQA

SEQ ID NO: 5 - protein, 45 aa, Artificial Sequence
  Description of Artificial Sequence Synthetic polypeptide
  CD8a hinge domain
TSTPAPRPPTPAPTIASQPLSLRPEACRPAAGGAVHTRGLDFACD

SEQ ID NO: 6 - protein, 262 aa, Artificial Sequence
  Description of Artificial Sequence Synthetic polypeptide
  GC5B81_LH extracellular domain
MAWVWTLLFLMAAAQSIQADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSS

SEQ ID NO: 7 - protein, 262 aa, Artificial Sequence
  Description of Artificial Sequence Synthetic polypeptide
  GC5B81_HL extracellular domain
MAWVWTLLFLMAAAQSIQAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 8 - protein, 264 aa, Artificial Sequence
  Description of Artificial Sequence Synthetic polypeptide
  BCMB69_HL extracellular domain
MAWVWTLLFLMAAAQSIQAQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 9 - protein, 264 aa, Artificial Sequence
  Description of Artificial Sequence Synthetic polypeptide
  BCMB69_LH extracellular domain
MAWVWTLLFLMAAAQSIQASYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGTEGKSSGSGSESKSTQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 10 - protein, 261 aa, Artificial Sequence
  Description of Artificial Sequence Synthetic polypeptide
  I3RB135_LH extracellular domain
MAWVWTLLFLMAAAQSIQAEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSS

SEQ ID NO: 11 - protein, 261 aa, Artificial Sequence
  Description of Artificial Sequence Synthetic polypeptide
  I3RB135_HL extracellular domain
MAWVWTLLFLMAAAQSIQAEVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 12 - protein, 267 aa, Artificial Sequence
  Description of Artificial Sequence Synthetic polypeptide
  CD33B904_LH extracellular domain
MAWVWTLLFLMAAAQSIQADIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKGTEGKSSGSGSESKSTEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSS

SEQ ID NO: 13 - protein, 267 aa, Artificial Sequence
  Description of Artificial Sequence Synthetic polypeptide
  CD33B904_HL extracellular domain
MAWVWTLLFLMAAAQSIQAEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSGTEGKSSGSGSESKSTDIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIK

SEQ ID NO: 14 - protein, 243 aa, Artificial Sequence
  Description of Artificial Sequence Synthetic polypeptide
  GC5B81_LH scFv
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSS

SEQ ID NO: 15 - protein, 243 aa, Artificial Sequence
  Description of Artificial Sequence Synthetic polypeptide
  GC5B81_HL scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 16 - protein, 245 aa, Artificial Sequence
  Description of Artificial Sequence Synthetic polypeptide
  BCMB69_HL scFv
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 17 - protein, 245 aa, Artificial Sequence
  Description of Artificial Sequence Synthetic polypeptide
  BCMB69_LH scFv
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGTEGKSSGSGSESKSTQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 18 - protein, 242 aa, Artificial Sequence
  Description of Artificial Sequence Synthetic polypeptide
  I3RB135_LH scFv
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSS

SEQ ID NO: 19 - protein, 242 aa, Artificial Sequence
  Description of Artificial Sequence Synthetic polypeptide
  I3RB135_HL scFv
EVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 20 - protein, 248 aa, Artificial Sequence
  Description of Artificial Sequence Synthetic polypeptide
  CD33B904_LH scFv
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKGTEGKSSGSGSESKSTEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSS

SEQ ID NO: 21 - protein, 248 aa, Artificial Sequence
  Description of Artificial Sequence Synthetic polypeptide
  CD33B904_HL scFv
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSGTEGKSSGSGSESKSTDIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIK

SEQ ID NO: 22 - protein, 485 aa, Artificial Sequence
  Description of Artificial Sequence Synthetic polypeptide
  GC5B81_LH CAR
MAWVWTLLFLMAAAQSIQADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 485 aa, Artificial Sequence
  Description of Artificial Sequence Synthetic polypeptide
  GC5B1_HL CAR
MAWVWTLLFLMAAAQSIQAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 487 aa, Artificial Sequence
  Description of Artificial Sequence Synthetic polypeptide
  BCMB69_HL CAR
MAWVWTLLFLMAAAQSIQAQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 487 aa, Artificial Sequence
  Description of Artificial Sequence Synthetic polypeptide
  BCMB69_LH CAR
MAWVWTLLFLMAAAQSIQASYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGTEGKSSGSGSESKSTQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 484 aa, Artificial Sequence
  Description of Artificial Sequence Synthetic polypeptide
  I3RB135_LH CAR
MAWVWTLLFLMAAAQSIQAEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 484 aa, Artificial Sequence
  Description of Artificial Sequence Synthetic polypeptide
  I3RB135_HL CAR
MAWVWTLLFLMAAAQSIQAEVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 490 aa, Artificial Sequence
  Description of Artificial Sequence Synthetic polypeptide
  CD33B904_LH CAR
MAWVWTLLFLMAAAQSIQADIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKGTEGKSSGSGSESKSTEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 490 aa, Artificial Sequence
  Description of Artificial Sequence Synthetic polypeptide
  CD33B904_HL CAR
MAWVWTLLFLMAAAQSIQAEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSGTEGKSSGSGSESKSTDIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1455 bp, Artificial Sequence
  Description of Artificial Sequence Synthetic polynucleotide
  GC5B81_LH CAR
atggcttgggtgtggaccttgctattcctgatggcagctgcccaaagtatacaggccgacatccagatgacccagagcccaagcagcctgagcgccagcgtgggcgaccgcgtaaccattacttgtcgggcttcccaaagcattagtagctatttgaattggtatcaacaaaaaccaggcaaggccccaaagctgctgatctacgccgcctctagcctgcagagcggagtgccaagccgcttcagcggcagcggcagcggcaccgacttcaccctgaccatcagcagcctgcagccagaggacttcgccacctactactgccagcagagctacagcaccccactgaccttcggccagggcaccaaggtggagatcaagggtacagagggcaagtctagtggaagtggtagcgaaagtaagagtacccaggtgcagctggtgcagagcggcgccgaggtgaagaagccaggcagcagcgtgaaggtgagctgcaaggccagcggcggcaccttcagcagctacgccatcagctgggtgcgccaggccccaggccagggactggagtggatgggcggcatcatcccaatcttcggcaccgccaactacgcccagaagttccagggccgcgtgaccatcaccgccgacgagagcaccagcaccgcctacatggagctgagcagcctgcgcagcgaggacaccgccgtgtactactgcgcacgcgagagccgctggcgcggctacaagctggactactggggccagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 31 - DNA, 1455 bp, Artificial Sequence
  Description of Artificial Sequence Synthetic polynucleotide
  GC5B81_HL CAR
atggcttgggtgtggaccttgctattcctgatggcagctgcccaaagtatacaggcccaggtgcagctggtgcagagcggcgccgaggtgaagaagccaggcagcagcgtgaaggtgagctgcaaggccagcggcggcaccttcagcagctacgccatcagctgggtgcgccaggccccaggccagggactggagtggatgggcggcatcatcccaatcttcggcaccgccaactacgcccagaagttccagggccgcgtgaccatcaccgccgacgagagcaccagcaccgcctacatggagctgagcagcctgcgcagcgaggacaccgccgtgtactactgcgcacgcgagagccgctggcgcggctacaagctggactactggggccagggcaccctggtgaccgtgagcagcggtacagagggcaagtctagtggaagtggtagcgaaagtaagagtaccgacatccagatgacccagagcccaagcagcctgagcgccagcgtgggcgaccgcgtaaccattacttgtcgggcttcccaaagcattagtagctatttgaattggtatcaacaaaaaccaggcaaggccccaaagctgctgatctacgccgcctctagcctgcagagcggagtgccaagccgcttcagcggcagcggcagcggcaccgacttcaccctgaccatcagcagcctgcagccagaggacttcgccacctactactgccagcagagctacagcaccccactgaccttcggccagggcaccaaggtggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 32 - DNA, 1461 bp, Artificial Sequence
  Description of Artificial Sequence Synthetic polynucleotide
  BCMB69_HL CAR
atggcttgggtgtggaccttgctattcctgatggcagctgcccaaagtatacaggcccagctgcaattgcaggaatccggtcctggactcgtaaagcctagcgagactttgagcctgacttgcactgtgtcaggtggctctatttctagtgggagttatttttggggctggatacgccagccacctggaaaggggttggagtggattgggtccatctactattctggaattacatactataacccttccttgaaatctcgcgttaccatcagtgtagatacctctaaaaaccaattctcccttaaacttagtagcgttaccgccgcagatacagctgtctattattgcgcccgtcatgacggcgcagtagctggtctcttcgactattggggccaaggcaccttggtcacagtttctagcggtacagagggcaagtctagtggaagtggtagcgaaagtaagagtaccagctacgtcctgacacaacccccttctgtctctgtagctcccggtcaaaccgctcggatcacttgtggaggcaataatatcgggagcaagtcagtgcattggtatcagcagccccctggtcaggccccagttgtggtagtctatgatgattccgaccgcccaagcggcatacccgagcgttttagtggcagtaattctggtaacacagcaactcttactatctctcgcgttgaagcaggcgacgaagcagtatattattgccaggtatgggactcttctagtgatcatgtggtgttcggcggtggcaccaagctgacagtgctgactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 33 - DNA, 1461 bp, Artificial Sequence
  Description of Artificial Sequence Synthetic polynucleotide
  BCMB69_LH CAR
atggcttgggtgtggaccttgctattcctgatggcagctgcccaaagtatacaggccagctacgtcctgacacaacccccttctgtctctgtagctcccggtcaaaccgctcggatcacttgtggaggcaataatatcgggagcaagtcagtgcattggtatcagcagccccctggtcaggccccagttgtggtagtctatgatgattccgaccgcccaagcggcatacccgagcgttttagtggcagtaattctggtaacacagcaactcttactatctctcgcgttgaagcaggcgacgaagcagtatattattgccaggtatgggactcttctagtgatcatgtggtgttcggcggtggcaccaagctgacagtgctgggtacagagggcaagtctagtggaagtggtagcgaaagtaagagtacccagctgcaattgcaggaatccggtcctggactcgtaaagcctagcgagactttgagcctgacttgcactgtgtcaggtggctctatttctagtgggagttatttttggggctggatacgccagccacctggaaaggggttggagtggattgggtccatctactattctggaattacatactataacccttccttgaaatctcgcgttaccatcagtgtagatacctctaaaaaccaattctcccttaaacttagtagcgttaccgccgcagatacagctgtctattattgcgcccgtcatgacggcgcagtagctggtctcttcgactattggggccaaggcaccttggtcacagtttctagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 34 - DNA, 1452 bp, Artificial Sequence
  Description of Artificial Sequence Synthetic polynucleotide
  I3RB135_LH CAR
atggcttgggtgtggaccttgctattcctgatggcagctgcccaaagtatacaggccgagatcgtgctgacccagagccctgcaactctctccctgagtccaggtgagcgggctactctgtcatgtcgggcatcacagtctgtgagcagctacctggcctggtaccagcagaagccaggccaggccccacgcctgctgatctacgacgccagcaaccgcgccaccggcatcccagcccgcttcagcggcagcggcagcggcaccgacttcaccctgaccatcagcagcctggagccagaggacttcgccgtgtactactgccagcagcgcagcaactggccactgaccttcggccagggcaccaaggtggagatcaagggtacagagggcaagtctagtggaagtggtagcgaaagtaagagtaccgaggtgcagctgctggagagcggcggcggcctggtgcagccaggcggcagcctgcgcctgagctgcgccgccagcggcttcaccttcagcggctactggatgcactgggtgcgccaggccccaggcaagggcctggagtgggtgagcgccatccgcagcgacggcagcagcaagtactacgccgacagcgtgaagggccgcttcaccatcagccgcgacaacagcaagaacaccctgtacctgcagatgaacagcctgcgcgccgaggacaccgccgtgtactactgcgccaaggacggcgtgatcgaggacaccttcgactactggggccagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 35 - DNA, 1452 bp, Artificial Sequence
  Description of Artificial Sequence Synthetic polynucleotide
  I3RB135_HL CAR
atggcttgggtgtggaccttgctattcctgatggcagctgcccaaagtatacaggccgaggtgcagctgctggagagcggcggtggcttggtacaacccggtggatctctgcgattgagttgtgctgcctcaggtttcacttttagcggctactggatgcactgggtgcgccaggccccaggcaagggcctggagtgggtgagcgccatccgcagcgacggcagcagcaagtactacgccgacagcgtgaagggccgcttcaccatcagccgcgacaacagcaagaacaccctgtacctgcagatgaacagcctgcgcgccgaggacaccgccgtgtactactgcgccaaggacggcgtgatcgaggacaccttcgactactggggccagggcaccctggtgaccgtgagcagcggtacagagggcaagtctagtggaagtggtagcgaaagtaagagtaccgagatcgtgctgacccagagcccagccaccctgagcctgagcccaggcgagcgcgccaccctgagctgccgcgccagccagagcgtgagctcatacttggcctggtatcagcagaaacccggacaagcacccagacttctcatatacgacgccagcaaccgcgccaccggcatcccagcccgcttcagcggcagcggcagcggcaccgacttcaccctgaccatcagcagcctggagccagaggacttcgccgtgtactactgccagcagcgcagcaactggccactgaccttcggccagggcaccaaggtggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 36 - DNA, 1470 bp, Artificial Sequence
  Description of Artificial Sequence Synthetic polynucleotide
  CD33B904_LH CAR
atggcttgggtgtggaccttgctattcctgatggcagctgcccaaagtatacaggccgacatcgtgatgacccagagcccagacagcctggccgtgagcctgggcgagcgcgccaccatcaactgcaagagcagccagaccgtgttctacagcagcaacaacaagaactacctggcctggtaccagcagaagccaggccagccaccaaagctgctgatcagctgggccagcacccgcaagagcggcgtgccagaccgcttcagcggcagcggcagcggcaccgacttcaccctgaccgtgagcagcctgcaggccgaggacgtggccgtgtactactgccagcactactacagcaccccatacaccttcggccagggcaccaagctggagatcaagggtacagagggcaagtctagtggaagtggtagcgaaagtaagagtaccgaggtgcagctggtggagagcggcggcggcctggtgcagccaggccgcagcctgcgcctgagctgcgccgccagcggcttcaccttcgacgactacgccatgcactgggtgcgccaggccccaggcaagggcctggagtgggtgagcggcatcggctggagcggcggcagcatcgtgtacgccgacagcgtgaagggccgcttcaccatcagccgcgacaacgccaagaacagcctgtacctgcagatgaacagcctgcgcgccgaggacaccgccctgtactactgcgccaaggacagcccatacggcgacttcttcgactactggggccagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 37 - DNA, 1470 bp, Artificial Sequence
  Description of Artificial Sequence Synthetic polynucleotide
  CD33B904_HL CAR
atggcttgggtgtggaccttgctattcctgatggcagctgcccaaagtatacaggccgaggtgcagctggtggagagcggcggcggcctggtgcagccaggccgcagcctgcgcctgagctgcgccgccagcggcttcaccttcgacgactacgccatgcactgggtgcgccaggccccaggcaagggcctggagtgggtgagcggcatcggctggagcggcggcagcatcgtgtacgccgacagcgtgaagggccgcttcaccatcagccgcgacaacgccaagaacagcctgtacctgcagatgaacagcctgcgcgccgaggacaccgccctgtactactgcgccaaggacagcccatacggcgacttcttcgactactggggccagggcaccctggtgaccgtgagcagcggtacagagggcaagtctagtggaagtggtagcgaaagtaagagtaccgacatcgtgatgacccagagcccagacagcctggccgtgagcctgggcgagcgcgccaccatcaactgcaagagcagccagaccgtgttctacagcagcaacaacaagaactacctggcctggtaccagcagaagccaggccagccaccaaagctgctgatcagctgggccagcacccgcaagagcggcgtgccagaccgcttcagcggcagcggcagcggcaccgacttcaccctgaccgtgagcagcctgcaggccgaggacgtggccgtgtactactgccagcactactacagcaccccatacaccttcggccagggcaccaagctggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

(19) United States
(12) Patent Application Publication
(10) Pub. No.: US 2020/0268797 A1
(43) Pub. Date: Aug. 27, 2020

(54) GAMMA DELTA T CELLS AND USES THEREOF

(71) Applicant: Janssen Biotech, Inc., Horsham, PA (US)

(72) Inventors: Rajkumar GANESAN, Blue Bell, PA (US); Iqbal S. GREWAL, Newtown, PA (US); Sanjaya SINGH, Blue Bell, PA (US)

(21) Appl. No.: 16/691,812

(22) Filed: Nov. 22, 2019

Related U.S. Application Data
(60) Provisional application No. 62/773,594, filed on Nov. 30, 2018.

(51) Int. Cl. A61K 35/17; C12N 5/0783; C12N 13/00; C07K 16/30; C07K 14/705; C07K 14/725

(52) U.S. Cl. CPC A61K 35/17; C07K 2317/73; C12N 13/00; C07K 16/30; C07K 14/70517; C07K 14/7051; C12N 2506/11; C12N 2501/999; C12N 2501/2302; C12N 2501/2315; C07K 2317/24; C07K 2319/03; C07K 2319/33; C07K 2319/02; C07K 2317/53; C12N 5/0636

(57) ABSTRACT

Provided are methods of expanding and isolating γδ T cells from human peripheral blood mononuclear cells (PBMCs). Also provided are isolated γδ T cells, CAR-γδ T cells, and methods of using the same.

### RELATED APPLICATIONS

This application claims the benefit of U.S. Provisional Application Ser. No. 62/773,594, filed 30 Nov. 2018, the entire contents of the aforementioned application is incorporated herein by reference in the entirety.

### FIELD OF THE INVENTION

This invention relates to the expansion and isolation of γδ T cells and the use of the γδ T cells to express a chimeric antigen receptor for adoptive T cell therapy.

### BACKGROUND OF THE INVENTION

The genetic engineering of T cells to specifically engage and kill tumor cells in a target-specific manner has resulted in the establishment of new therapeutic options for cancer patients, referred to as engineered T cell therapy. This targeting is typically brought about by genetically manipulating patient-derived T cells with a recombinant DNA molecule that encodes a chimeric antigen receptor (CAR). CARs are synthetic receptors comprising an extracellular targeting domain that is linked to a linker peptide, a transmembrane (TM) domain, and one or more intracellular signaling domains. Traditionally, the extracellular domain consists of a single chain Fv fragment of an antibody (scFv) that is specific for a given tumor-associated antigen (TAA) or cell surface target. The extracellular scFv domain confers the tumor specificity of the CAR, while the signaling domains activate the T cell upon TAA/target engagement. These engineered T cells (CAR-T cells) are re-infused into cancer patients, where they specifically engage and kill cells expressing the TAA target of the CAR (Maus et al., Blood. 2014 Apr. 24; 123(17):2625-35; Curran and Brentjens, J Clin Oncol. 2015 May 20; 33(15):1703-6).

Autologous, patient-specific CAR-T therapy has emerged as a powerful and potentially curative therapy for cancer, especially for CD19-positive hematological malignancies. However, the development of CAR-T technology and its wider application is partly limited due to number of key shortcomings including a) an inefficient anti-tumor response in solid tumors, b) limited penetration and susceptibility of adoptively transferred CAR T cells to an immunosuppressive tumor microenvironment (TME), c) poor persistence of CAR-T cells in vivo, d) serious adverse events in the patients including cytokine release syndrome (CRS) and graft-versus-host disease (GVHD) mediated by the CAR-T, and e) the time required for manufacturing.

To circumvent major issues with the current CAR-T approaches, alternative CAR-T strategies should be developed.

### BRIEF SUMMARY OF THE INVENTION

In one general aspect, provided is a method of expanding and isolating γδ T cells from human peripheral blood mononuclear cells (PBMCs). The methods comprise (a) obtaining human PBMCs; (b) culturing the human PBMCs in a culture media comprising zoledronic acid, interleukin-2 (IL-2), and interleukin-15 (IL-15) to expand the γδ T cells; and (c) isolating the γδ T cells. In certain embodiments, the concentration of the zoledronic acid is about 1 μM to about 20 μM. In certain embodiments, the concentration of the zoledronic acid is about 5 μM.

In certain embodiments, the concentration of the IL-2 is about 50 IU/mL to about 5000 IU/mL. The concentration of IL-2 can, for example, be about 100 IU/mL to about 1000 IU/mL. In certain embodiments, the IL-2 is recombinant human IL-2 (rhIL-2).

In certain embodiments, the concentration of IL-15 is about 1 ng/mL to about 100 ng/mL. The concentration of IL-15 can, for example, be about 10 ng/mL. In certain embodiments, the IL-15 is recombinant human IL-15 (rhIL-15).

In certain embodiments, the γδ T cell is a Vγ9Vδ2 T cell. In certain embodiments, the γδ T cells are isolated by flow cytometry, magnetic separation, and negative selection.

Also provided are isolated γδ T cells produced by the methods of the invention.

Also provided are methods of generating a chimeric antigen receptor (CAR)-γδ T cell. The methods comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

In certain embodiments, the CAR comprises (i) an extracellular domain comprising an antigen binding domain and/or an antigen binding fragment; (ii) a transmembrane domain comprising a CD8α transmembrane domain; (iii) an intracellular signaling domain comprising a CD3ζ or 4-1BB intracellular domain; (iv) a signal peptide comprising a CD8α signal peptide; and (v) a hinge region comprising a CD8α hinge region.

In certain embodiments, the CAR comprises (i) the transmembrane domain having an amino acid sequence at least 90% identical to SEQ ID NO:1; (ii) the intracellular domain having an amino acid sequence at least 90% identical to SEQ ID NO:2 or SEQ ID NO:3; (iii) the signal peptide having an amino acid sequence at least 90% identical to SEQ ID NO:4; and (iv) the hinge region having an amino acid sequence at least 90% identical to SEQ ID NO:5.

In certain embodiments, the extracellular domain comprises an antigen binding domain and/or an antigen binding fragment that specifically binds a tumor antigen.

In certain embodiments, the CAR comprises an amino acid sequence selected from the group consisting of SEQ ID NOs:22-29.

Also provided are CAR-γδ T cells produced by the methods of the invention.

In certain embodiments, provided is a pharmaceutical composition comprising the CAR-γδ T cell of the invention and a pharmaceutically acceptable carrier.

Also provided are methods of treating or preventing a disease or condition in a subject in need thereof. The methods comprise administering a therapeutically effective amount of the pharmaceutical composition of the invention. In certain embodiments, the disease or condition is cancer. The cancer can, for example, be selected from a solid cancer or a liquid cancer. The cancer can be, but is not limited to, a cancer selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

In certain embodiments, the disease or condition is an autoimmune disease. The autoimmune disease can be, but is not limited to, an autoimmune disease selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere;s disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

Also provided are methods of producing a pharmaceutical composition comprising a CAR-γδ T cell, wherein the methods comprises combining the CAR-γδ T cell of the invention with a pharmaceutically acceptable carrier to obtain the pharmaceutical composition.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows distribution of Vγ9⁺γδ T cells among total PBMCs. Total PBMCs were Fc blocked and stained with anti-TCR Vγ9 and Vδ2 antibodies. Representative FACS plot shows the frequency of live Vγ9⁺, Vγ9⁺Vδ2⁺, and Vδ2⁺γδ T cells among total PBMCs.

FIGS. 2A-2C show Zol selectively expands of Vγ9⁺γδ T cells from whole PBMCs. Whole PBMCs were cultured in complete RPMI (+10% FBS+1×Pen/Strep) supplemented with either rhIL-2+rhIL-15 or Zol+rhIL-2+rhIL-15 for various time points up to 14 days. FIG. 2A shows the flow cytometry gating strategy for identifying TCR Vγ9⁺γδ T cells. FIG. 2B shows FACS plots that represent the frequency of Vγ9⁺ cells among whole PBMCs cultured in the absence or presence of Zol for 14 days. Gated cells represent the frequency of Vγ9⁺ cells among total PBMCs. FIG. 2C shows the absolute number of Vγ9⁺ cells among whole PBMCs that were cultured with rhIL-2+rhIL-15 or with Zol+rhIL-2+rhIL-15 for 8, 12 and 14 days. Light and dark bars represent PBMCs cultured in complete RPMI medium containing rhIL-2+rhIL-15 and Zol+rhIL-2+rhIL-15 respectively. Data represented here is cumulative from two donors, HPU-06517 and HPU-11073.

FIGS. 3A-3C show the phenotype of resting and activated Vγ9⁺γδ T cells. Resting and Zol activated Vγ9⁺γδ T cells were surface stained with CD45RA, CD27, CD69, CD44, PD1, Tim-3, 2B4, Lag3 and CTLA-4. FIG. 3A shows the differentiation profile of Vγ9⁺γδ T cells from fresh PBMCs (left) and Zol activated (right). Numbers in each quadrant represent the frequency of the population respective to the gate. FIG. 3B shows the activation and effector profile of fresh and activated Vγ9⁺γδ T cells. CD44 and CD69 surface expression was measured, while Granzyme B and Perforin were profiled intracellularly. FIG. 3C shows PD1, Tim-3, 2B4, Lag3 and CTLA-4 expression on fresh and activated Vγ9⁺γδ T cells. Numbers in each FACS plot represent the median fluorescence intensity (MFI) values for the respective antibody. Filled black colored closed histogram indicates FMO control for respective staining. Open histogram shows the expression of respective marker on Vγ9⁺γδ T cells.

FIG. 4 shows enriching γδ T cells via negative selection. Day14 cultured γδ T cells were enriched via negative selection using EasySep Human γδ T cell isolation kit. Representative histograms show the frequency of TCRαβ and TCRγδ before enrichment (left) and after enrichment (right). Numbers in the gate refer to the frequency cells among total cells.

FIGS. 5A and 5B show cell viability and transfection efficiency of enriched γδ T cells transfected with CAR mRNA. Day14 enriched γδ T cells were electroporated (1400V, 20 ms pulse width, 1 pulse) with either GFP or I3RB135_LH or I3RB135-HL CAR mRNA on a Neon transfection system. FIG. 5A shows representative FACS plots that depict the frequency of live cells among total γδ T cells (upper row) and the frequency of GFP⁺ cells (bottom row, left panel) or CD123⁺ cells (bottom row, middle and right panels) among total live γδ T cells. FIG. 5B, Day14 enriched γδ T cells were cultured with either GFP or I3RB135 LH or I3RB135-HL CAR mRNA without transfection system and shows representative FACS plots that depict the frequency of live cells among total γδ T cells (upper row) and the frequency of GFP+ cells (bottom row, left panel) or CD123+ cells (bottom row, middle and right panels) among total live γδ T cells.

FIGS. 6A-6E show the cytotoxicity profile of CAR transfected γδ T cells. Day14 enriched γδ T cells were electroporated (1400V, 20 ms, 1 pulse) with either EGFP or various tumor-targeting CAR mRNA constructs. Target expressing tumor cell line (Wt) labelled with CellTracker™ Green CMFDA Dye was mixed in 1:1 ratio with target ablated isogenic knock out tumor cell lines (KO) labelled with CellTracker™ Orange CMRA Dye and co-culture with CAR Transfected γδ T cells at various ET ratios. After 20 hrs of co-culture, cytotoxicity of tumor cells was measured by staining with 7-AAD. FIG. 6A shows schematic representation of γδ CAR T cell selective cytotoxicity against TAA expressing cell lines. FIG. 6B shows BCMA γδ CAR-T cell mediated selective cytotoxicity against BCMA expressing target cell line. FIG. 6C shows GPRC5D γδ CAR-T cell mediated selective cytotoxicity against GPRC5D expressing H929 cell line. FIG. 6D shows CD33 γδ CAR-T cell mediated selective cytotoxicity against CD33 expressing target cell line. FIG. 6E shows anti-CD123 γδ CAR-T cell mediated selective cytotoxicity against CD123+ expressing target cell line while sparing the CD123− cells.

FIG. 7 shows a schematic of an anti-tumor associated antigen (TAA) CAR-T construct composed of an antigen specific single chain Fv (scFv) followed a flexible hinge sequence and a transmembrane segment (from human CD8). The cytoplasmic region was composed of 4-1BB and CD3ζ.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2(4):482-489 (1981), by the homology alignment algorithm of Needleman & Wunsch, J Mol. Biol. 48(3):443-453 (1970), by the search for similarity method of Pearson & Lipman, Proc. Nat'l. Acad. Sci. USA 85(8):2444-2448 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and

RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell (e.g., a γδ T cell), a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture, or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells (e.g., γδ T cells), and are referred to as CAR-T cells (e.g., CAR γδ T cells) because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention.

Methods of Expanding and Purifying γδ T Cells

Provided herein are γδ T cell-based allogenic off-the-shelf CAR-T cell products. Use of γδ T cells allows for the development of a high-quality product combining the inherent versatile nature of the γδ T cell with their highly potent cytolytic functions to reduce the risk of tumor escape. This approach can help to reduce the side effects mediated by CRS/GVHD and prevent long-term autoimmunity while providing excellent efficacy, particularly, in solid tumors. γδ T cells are abundant in the blood with good markers for sorting and can easily be activated and expanded in large numbers by well-defined ligands. Since γδ T cell recognition is independent of MHC, γδ T cells do not participate in GVHD, and there is no risk of allogeneic-recognition, γδ T cells can serve as an allogenic source of CAR-T for a broader population of patients. Off the shelf products can have several advantages including consistent quality, cells can be sorted for 100% transductions, no limitations on the dose size, reduced cost and significant time savings for the patients to initiate the treatment.

According to particular aspects, the invention provides methods of expanding and isolating γδ T cells from human peripheral blood mononuclear cells (PBMCs). In one general aspect, the methods comprise (a) obtaining human PBMCs; (b) culturing the human PBMCs in a culture media comprising zoledronic acid, interleukin-2 (IL-2), and interleukin-15 (IL-15) to expand the γδ T cells; and (c) isolating the γδ T cells. In certain embodiments, the γδ T cell is a Vγ9Vδ2 T cell.

Also provided are isolated γδ T cells, including isolated Vγ9Vδ2 T cells, produced by the methods of the invention.

In certain embodiments, the concentration of the zoledronic acid is about 1 μM to about 20 μM. The concentration of zoledronic acid can be about 3 μM to about 18 μM, about 5 μM to about 16 μM, about 7 μM to about 14 μM, about 9 μM to about 12 μM. The concentration of zoledronic acid can, for example, be about 1, 2, 3, 4, 5, 6, 7, 8, 9, 10, 11, 12, 13, 14, 15, 16, 17, 18, 19, or 20 μM. In certain embodiments, the concentration of the zoledronic acid is about 5 μM.

In certain embodiments, the concentration of the IL-2 is about 50 IU/mL to about 5000 IU/mL. The concentration of IL-2 can, for example, be about 50 IU/mL to about 4000 IU/mL, about 50 IU/mL to about 3000 IU/mL, about 50 IU/mL to about 2000 IU/mL, about 50 IU/mL to about 1000 IU/mL, about 50 IU/mL to about 500 IU/mL, about 50 IU/mL to about 250 IU/mL, about 100 IU/mL to about 5000 IU/mL, about 100 IU/mL to about 4000 IU/mL, about 100 IU/mL to about 3000 IU/mL, about 100 IU/mL to about 2000 IU/mL, about 100 IU/mL to about 1000 IU/mL, about 100 IU/mL to about 500 IU/mL, about 250 IU/mL to about 5000 IU/mL, about 250 IU/mL to about 4000 IU/mL, about 250 IU/mL to about 3000 IU/mL, about 250 IU/mL to about 2000 IU/mL, about 250 IU/mL to about 1000 IU/mL, about 500 IU/mL to about 5000 IU/mL, about 500 IU/mL to about 4000 IU/mL, about 500 IU/mL to about 3000 IU/mL, about 500 IU/mL to about 2000 IU/mL, about 500 IU/mL to about 1000 IU/mL, about 1000 IU/mL to about 5000 IU/mL, about 1000 IU/mL to about 4000 IU/mL, about 1000 IU/mL to about 3000 IU/mL, about 1000 IU/mL to about 2000 IU/mL, about 2000 IU/mL to about 5000 IU/mL, about 2000 IU/mL to about 4000 IU/mL, about 2000 IU/mL to about 3000 IU/mL, or any concentration in between. In certain embodiments, the concentration of IL-2 is 50, 100, 250, 500, 1000, 1500, 2000, 2500, 3000, 3500, 4000, 4500, or 5000 IU/mL. In certain embodiments, the IL-2 is recombinant human IL-2 (rhIL-2).

In certain embodiments, the concentration of IL-15 is about 1 ng/mL to about 100 ng/mL. The concentration of IL-15 can be about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, about 1 ng/mL to about 10 ng/mL, about 10 ng/mL to about 100 ng/mL, about 10 ng/mL to about 90 ng/mL, about 10 ng/mL to about 80 ng/mL, about 10 ng/mL to about 70 ng/mL, about 10 ng/mL to about 60 ng/mL, about 10 ng/mL to about 50 ng/mL, about 10 ng/mL to about 40 ng/mL, about 10 ng/mL to about 30 ng/mL, about 10 ng/mL to about 20 ng/mL, about 25 ng/mL to about 100 ng/mL, about 25 ng/mL to about 90 ng/mL, about 25 ng/mL to about 80 ng/mL, about 25 ng/mL to about 70 ng/mL, about 25 ng/mL to about 60 ng/mL, about 25 ng/mL to about 50 ng/mL, about 25 ng/mL to about 40 ng/mL, about 50 ng/mL to about 100 ng/mL, about 50 ng/mL to about 90 ng/mL, about 50 ng/mL to about 80 ng/mL, about 50 ng/mL to about 70 ng/mL, about 50 ng/mL to about 60 ng/mL, or any value in between. The concentration of IL-15 can, for example, be about 10 ng/mL. In certain embodiments, the IL-15 is recombinant human IL-15 (rhIL-15).

Also provided are methods of generating a chimeric antigen receptor (CAR)-γδ T cell. The methods comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR-γδ T cell.

Thus, in certain embodiments, the isolated γδ T cells can comprise an isolated polynucleotide encoding a CAR or a vector comprising the isolated polynucleotide encoding the CAR. The immune cells comprising the isolated polynucleotides and/or vectors can be referred to as “engineered immune cells.” Preferably, the engineered immune cells are derived from a human (are of human origin prior to being made recombinant). The engineered immune cells can, for example, be T cells, and in particular are γδ T cells isolated by the methods described herein. In certain embodiments, the γδ T cells are Vγ9Vδ2 T cells isolated by the methods described herein.

γδ T cells, including Vγ9Vδ2 T cells, can be expanded and isolated utilizing the methods disclosed herein. Immune cells can additionally be isolated by methods known in the art, including commercially available methods (see, e.g., Rowland Jones et al., Lymphocytes: A Practical Approach, Oxford University Press, NY (1999)). Sources for immune cells or precursors thereof include, but are not limited to, peripheral blood (e.g., peripheral blood mononuclear cells (PBMCs)), umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolated or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells. In certain embodiments, the γδ T cells are isolated by flow cytometry, magnetic separation, and negative selection.

The γδ T cells can be autologous or non-autologous to the subject to which they are administered in the methods of treatment of the invention. Autologous cells are isolated from the subject to which the engineered immune cells recombinantly expressing the CAR are to be administered. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as γδ T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-γ, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

Chimeric Antigen Receptors (CARs)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

As used herein, the term “intracellular T cell receptor-activating signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by a T cell that provides the primary cytoplasmic signaling sequence(s) that regulate primary activation of the T cell receptor (TCR) complex in a stimulatory way for at least some aspect of the T cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain general aspects, provided herein are methods of generating a chimeric antigen receptor (CAR) γδ T cell. The methods can comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

In certain embodiments, the extracellular domain comprises an antigen binding domain and/or an antigen binding fragment. In certain embodiments, the extracellular domain comprises an amino acid sequence at least 90% identical to an amino acid sequence selected from the group consisting of SEQ ID NOs:6-13, preferably an amino acid sequence selected from the group consisting of SEQ ID NOs:6-13.

In certain embodiments, the antigen binding fragment is a Fab, a Fab′, a F(ab′)2, an Fv, a single-chain variable fragment (scFv), a minibody, a diabody, a single-domain antibody (sdAb), a light chain variable domain (VL), or a variable domain (VHH) of a camelid antibody.

In certain embodiments, the antigen binding fragment is a single-chain variable fragment (scFv). The scFv can, for example, be an amino acid sequence selected from the group consisting of SEQ ID NOs:14-21.

In certain embodiments, the antigen binding domain and/or antigen binding fragment can, for example, specifically bind a tumor antigen. Any suitable tumor antigen for binding by an antibody or antigen binding fragment can be chosen based on the type of tumor and/or cancer exhibited by the subject to be treated.

In certain embodiments, the extracellular domain of the CAR is preceded by a signal peptide at the amino-terminus. Any suitable signal peptide can be used in the invention. The signal peptide can, for example, be derived from a natural, synthetic, semi-synthetic, or recombinant source. According to one embodiment, the signal peptide is a human CD8α signal peptide, a human CD3δ signal peptide, a human CD3ζ signal peptide, a human GMCSFR signal peptide, a human 4-1BB signal peptide, or a derivative thereof. According to particular embodiments, the signal peptide is a human CD8a signal peptide. The human CD8α signal peptide comprises an amino acid sequence at least 90% identical to SEQ ID NO:4, preferably the amino acid sequence of SEQ ID NO:4. The signal peptide can be cleaved by a signal peptidase during or after completion of translocation of the CAR to generate a mature CAR free of the signal peptide.

In certain embodiments, the CAR can further comprise a hinge region connecting the extracellular domain and the transmembrane domain. The hinge region functions to move the extracellular domain away from the surface of the engineered immune cell to enable proper cell/cell contact, binding to the target or antigen and activation (Patel et al., Gene Therapy 6:412-9 (1999)). Any suitable hinge region can be used in a CAR of the invention. The hinge region can be derived from a natural, synthetic, semi-synthetic, or recombinant source. According to particular embodiments, the hinge region of the CAR is a hinge region from a CD8α peptide. In particular embodiments, the hinge region comprises an amino acid sequence at least 90% identical to SEQ ID NO:5, preferably the amino acid sequence of SEQ ID NO:5.

A CAR of the invention comprises a transmembrane domain. Any suitable transmembrane domain can be used in a CAR of the invention. The transmembrane domain can be derived from a natural, synthetic, semi-synthetic, or recombinant source. According to some embodiments, the transmembrane domain is a transmembrane domain from a peptide selected from the group consisting of a CD8α peptide, a CD28 peptide, a CD4 peptide, a CD3ζ peptide, a CD2 peptide, a 4-1BB peptide, an OX40 peptide, an ICOS peptide, a CTLA-4 peptide, a PD-1 peptide, a LAG-3 peptide, a 2B4 peptide, a BTLA peptide, a GMCSFR peptide, and the like. In particular embodiments, the transmembrane domain is a CD8α transmembrane domain. The CD8α transmembrane domain can comprise an amino acid sequence at least 90% identical to SEQ ID NO:1, preferably the amino acid sequence of SEQ ID NO:1.

A CAR of the invention comprises an intracellular signaling domain. Any suitable intracellular domain can be used in a CAR of the invention. In particular embodiments, the entire intracellular signaling domain is used. In other particular embodiments, a truncated portion of the signaling domain that transduces the effector or signal is used. According to embodiments of the invention, the intracellular signaling domain generates a signal that promotes an immune effector function of the CAR-containing cell, e.g., a CAR-T cell, including, but not limited to, proliferation, activation, and/or differentiation. In particular embodiments, the signal promotes, e.g., cytolytic activity, helper activity, and/or cytokine secretion of the CAR-T cell. According to some embodiments, the intracellular signaling domain of the CAR comprises a signaling domain of an Fcγ receptor (FcγR), an FCC receptor (FcεR), an Fcα receptor (FcαR), neonatal Fc receptor (FcRn), CD3, CD3, CD3ζ, CD3γ, CD3ε, CD4, CD5, CD8, CD21, CD22, CD28, CD32, CD40L (CD154), CD45, CD66δ, CD79a, CD79β, CD80, CD86, CD278 (also known as ICOS), CD247ζ, CD247η, DAP10, DAP12, FYN, LAT, Lck, MAPK, MHC complex, NFAT, NF-κB, PLC-γ, iC3β, C3δγ, C3δ, and Zap70. According to some embodiments, the intracellular signaling domain is selected from the group consisting of a signaling domain of CD3ζ, FcRγ, FcRβ, CD3γ, CD3δ, CD3ε, CD5, CD22, CD79α, CD79β, and CD66δ. In particular embodiments, the intracellular domain is a CD3ζ or 4-1BB intracellular domain. The CD3ζ or 4-1BB intracellular domain can comprise an amino acid sequence at least 90% identical to SEQ ID NO:2 or 3, respectively, preferably the amino acid sequence of SEQ ID NO:2 or 3, respectively

According to particular embodiments, the intracellular signaling domain further comprises one or more co-stimulatory signaling domains. The co-stimulatory domain can, for example, comprise a signaling domain of a peptide selected from:

2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8α, CD8β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In certain embodiments, the costimulatory domain is selected from the group consisting of a costimulatory domain of one or more of CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

In certain embodiments, the CAR comprises an amino acid sequence selected from the group consisting of SEQ ID NOs:22-29.

Antigen-Binding Fragments

Antibodies

The invention generally relates to CAR constructs comprising an antigen binding fragment. The antigen binding fragment can, for example, be an antibody or antigen binding fragment thereof that specifically binds a tumor antigen. The antigen binding fragments of the invention possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

The antigen binding fragment can, for example, be an antibody or antigen binding fragment thereof that specifically binds a tumor antigen. Any suitable tumor antigen for binding by an antibody or antigen binding fragment can be chosen based on the type of tumor and/or cancer exhibited by the subject to be treated. Suitable antigens include, but are not limited to, mesothelin (MSLN), prostate specific membrane antigen (PSMA), prostate stem cell antigen (PCSA), carbonic anhydrase IX (CAIX), B-cell maturation antigen (BCMA or BCM), G-protein coupled receptor family C group 5 member D (GPRC5D), Interleukin-1 receptor accessory protein (IL1RAP), delta-like 3 (DLL3), carcinoembryonic antigen (CEA), CDS, CD7, CD10, CD19, CD20, CD22, CD30, CD33, CD34, CD38, CD41, CD44, CD49f, CD56, CD74, CD123, CD133, CD138, epithelial glycoprotein-2 (EGP 2), epithelial glycoprotein-40 (EGP-40), epithelial adhesion molecule (EpCAM), folate-binding protein (FBP), fetal acetylcholine receptor (AChR), folate receptor α and β (FRα and β), ganglioside G2 (GD2), ganglioside G3 (GD3), human epidermal growth factor receptor 2 (HER-2/ERB2), epidermal growth factor receptor (EGFR), epidermal growth factor receptor vIII (EGFRvIII), ERB3, ERB4, human telomerase reverse transcriptase (hTERT), interleukin-13 receptor subunit alpha-2 (IL-13Rα2), k-light chain, kinase insert domain receptor (KDR), Lewis A (CA19.9), Lewis Y (LeY), L1 cell adhesion molecule (LICAM), melanoma-associated antigen 1 (melanoma antigen family A1, MAGE-A1), Mucin-16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor receptor (VEGFR), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), chondroitin sulfate proteoglycan-4 (CSPG4), DNAX accessory molecule (DNAM-1), ephrin type A receptor 2 (EpHA2), fibroblast associated protein (FAP), Gp100/HLA-A2, glypican 3 (GPC3), HA-1H, HERK-V, IL-11Rα, latent membrane protein (LMP1), neural cell-adhesion molecule (N-CAM/CD56), and trail receptor (TRAIL R).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecifc antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on the tumor antigen and the second epitope is located on PD-1, PD-L1, CTLA-4, EGFR, HER-2, CD19, CD20, CD33, CD3, and/or other tumor associated immune suppressors or surface antigens.

As used herein, an antigen binding domain or antigen binding fragment that “specifically binds to a tumor antigen” refers to an antigen binding domain or antigen binding fragment that binds a tumor antigen, with a KD of 1×10⁻⁷ M or less, preferably 1×10⁻⁸M or less, more preferably 5×10⁻⁹ M or less, 1×10⁻⁹M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

The smaller the value of the KD of an antigen binding domain or antigen binding fragment, the higher affinity that the antigen binding domain or antigen binding fragment binds to a target antigen.

According to particular aspects, the invention relates to a CAR construct comprising an antigen binding fragment, wherein the antigen binding fragment is an antibody or antigen binding fragment that specifically binds a tumor antigen. The antibody or antigen binding fragment can, for example, be a Fab, a Fab′, a F(ab′)2, an Fv, a single-chain variable fragment (scFv), a minibody, a diabody, a single-domain antibody (sdAb), a light chain variable domain (VL), or a variable domain (VHH) of a camelid antibody.

Polynucleotides, Vectors, and Host Cells

In another general aspect, the invention relates to an isolated nucleic acid encoding a chimeric antigen receptor (CAR) of the invention. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARS of the invention can be altered without changing the amino acid sequences of the proteins.

In another general aspect, the invention relates to a vector comprising a CAR of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the invention.

In another general aspect, the invention relates to a host cell comprising a vector of the invention and/or an isolated nucleic acid encoding a CAR of the invention. Any host cell known to those skilled in the art in view of the present disclosure can be used for recombinant expression of CARs of the invention. Suitable host cells include prokaryotes, yeast, mammalian cells, or bacterial cells. In some embodiments, the host cells are E. coli TG1 or BL21 cells (for expression of, e.g., a CAR, a scFv, or sdAb), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Pharmaceutical Compositions

In another general aspect, the invention relates to a pharmaceutical composition comprising an isolated polynucleotide of the invention, an isolated polypeptide of the invention, a host cell of the invention, and/or an engineered immune cell of the invention and a pharmaceutically acceptable carrier. The term “pharmaceutical composition” as used herein means a product comprising an isolated polynucleotide of the invention, an isolated polypeptide of the invention, a host cell of the invention, and/or an engineered immune cell of the invention together with a pharmaceutically acceptable carrier. Polynucleotides, polypeptides, host cells, and/or engineered immune cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

Methods of Use

In another general aspect, the invention relates to a method of treating a disease or a condition in a subject in need thereof. The methods comprise administering to the subject in need thereof a therapeutically effective amount of an engineered immune cell and/or a pharmaceutical composition of the invention. In certain embodiments, the disease or condition is cancer. The cancer can, for example, be a solid or a liquid cancer.

The cancer, can, for example, be selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical composition comprises a therapeutically effective amount of an isolated polynucleotide, an isolated polypeptide, a host cell, and/or an engineered immune cell. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to an isolated polynucleotide, an isolated polypeptide, a host cell, an engineered immune cell, and/or a pharmaceutical composition of the invention a therapeutically effective amount means an amount of the isolated polynucleotide, the isolated polypeptide, the host cell, the engineered immune cell, and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention and/or the pharmaceutical compositions of the invention can be administered in any convenient manner known to those skilled in the art.

For example, the cells of the invention can be administered to the subject by aerosol inhalation, injection, ingestion, transfusion, implantation, and/or transplantation. The compositions comprising the cells of the invention can be administered transarterially, subcutaneously, intradermaly, intratumorally, intranodally, intramedullary, intramuscularly, inrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention and/or the pharmaceutical compositions of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition.

In a particular embodiment, “treat,” “treating,” and “treatment” refer to elimination of the disease, disorder, or condition in the subject.

### Embodiments

This invention provides the following non-limiting embodiments.

Embodiment 1 is a method of expanding and isolating γδ T cells from human peripheral blood mononuclear cells (PBMCs), the method comprising:
a. obtaining human PBMCs;
b. culturing the human PBMCs in a culture media comprising zoledronic acid, interleukin-2 (IL-2), and interleukin-15 (IL-15) to expand the γδ T cell; and
c. isolating the γδ T cells.

Embodiment 2 is the method of embodiment 1, wherein the concentration of the zoledronic acid is about 1 μM to about 20 μM.

Embodiment 3 is the method of embodiment 2, wherein the concentration of the zoledronic acid is about 5 μM.

Embodiment 4 is the method of any one of embodiment 1-3, wherein the concentration of the IL-2 is about 50 IU/mL to about 5000 IU/mL.

Embodiment 5 is the method of embodiment 4, wherein the concentration of IL-2 is about 100 IU/mL to about 1000 IU/mL.

Embodiment 6 is the method of any one of embodiments 1-5, wherein the IL-2 is recombinant human IL-2 (rhIL-2).

Embodiment 7 is the method of any one of embodiments 1-6, wherein the concentration of IL-15 is about 1 ng/mL to about 100 ng/mL.

Embodiment 8 is the method of embodiment 7, wherein the concentration of IL-15 is about 10 ng/mL.

Embodiment 9 is the method of any one of embodiments 1-8, wherein the IL-15 is recombinant human IL-15 (rhIL-15).

Embodiment 10 is the method of any one of embodiments 1-9, wherein the γδ T cell is a Vγ9Vδ2 T cell.

Embodiment 11 is the method of any one of embodiments 1-10, wherein the γδ T cells are isolated by flow cytometry, magnetic separation, and negative selection.

Embodiment 12 is an isolated γδ T cell produced by the method of embodiment 11.

Embodiment 13 is a method of generating a chimeric antigen receptor (CAR)-γδ T cell, the method comprising:
a. obtaining an isolated γδ T cell of embodiment 12;
b. contacting the γδ T cell with a nucleic acid encoding a chimeric antigen receptor (CAR), the CAR comprising:
   i. an extracellular domain;
   ii. a transmembrane domain; and
   iii. an intracellular signaling domain,
      wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

Embodiment 14 is the method of embodiment 13, wherein the CAR comprises:
i. an extracellular domain comprising an antigen binding domain and/or an antigen binding fragment;
ii. a transmembrane domain comprising a CD8α transmembrane domain;
iii. an intracellular signaling domain comprising a CD3ζ or 4-1BB intracellular domain;
iv. a signal peptide comprising a CD8α signal peptide; and
v. a hinge region comprising a CD8α hinge region.

Embodiment 15 is the method of embodiment 4, wherein the CAR comprises:
i. the transmembrane domain having an amino acid sequence at least 90% identical to SEQ ID NO:1;
ii. the intracellular domain having an amino acid sequence at least 90% identical to SEQ ID NO:2 or SEQ ID NO:3;
iii. the signal peptide having an amino acid sequence at least 90% identical to SEQ ID NO:4; and
iv. the hinge region having an amino acid sequence at least 90% identical to SEQ ID NO:5.

Embodiment 16 is the method of embodiment 14 or 15, wherein the extracellular domain comprises an antigen binding domain and/or an antigen binding fragment that specifically binds a tumor antigen.

Embodiment 17 is the method of any one of embodiments 13-16, wherein the CAR comprises an amino acid sequence selected from the group consisting of SEQ ID NOs:22-29.

Embodiment 18 is a CAR-γδ T cell produced by the method of any one of embodiments 13-17.

Embodiment 19 is a pharmaceutical composition comprising the CAR-γδ T cell of embodiment 18 and a pharmaceutically acceptable carrier.

Embodiment 20 is a method of treating or preventing a disease or condition in a subject in need thereof, the method comprising administering a therapeutically effective amount of the pharmaceutical composition of embodiment 19.

Embodiment 21 is the method of embodiment 20, wherein the disease or condition is cancer.

Embodiment 22 is the method of embodiment 21, wherein the cancer is selected from a solid cancer or a liquid cancer.

Embodiment 23 is the method of embodiment 22, wherein the cancer is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 24 is the method of embodiment 20, wherein the disease is an autoimmune disease.

Embodiment 25 is the method of embodiment 24, wherein the autoimmune disease is selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere;s disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

Embodiment 26 is a method of producing a pharmaceutical composition comprising a CAR-γδ T cell, wherein the methods comprises combining the CAR-γδ T cell of embodiment 18 with a pharmaceutically acceptable carrier to obtain the pharmaceutical composition.

### EXAMPLES

Materials and Methods

Reagents and Antibodies

TABLE 1
Reagents and Antibodies used in Examples

| | | Catalogue | |
|---|---|---|---|
| Item | Supplier | No | Lot number |
| RPMI 1640 | Gibco (Thermo | 11875-093 | 1924292 |
| | Fisher Scientific, | | |
| | Waltham, MA) | | |
| FBS | Gibco | 10099-141 | 1942645 |
| Penstrep | Gibco | 15070-063 | 1910854 |
| Recombinant Human | R&D systems | AE- | AE- |
| IL-2 Protein | (Minneapolis, | 6017031 | 6017031 |
| | MN) | | |
| FITC anti-human | Biolegend (San | 306014 | B201944 |
| CD123 Antibody | Diego, CA) | | |
| FITC Mouse IgG1, κ | Biolegend | 400110 | B206037 |
| Isotype Ctrl (FC) | | | |
| Antibody | | | |
| CFSE | Thermo Fisher | C34554 | 1878342 |
| | Scientific | | |
| | (Waltham, MA) | | |
| 7AAD | Biolegend | 420404 | B235875 |
| DPBS | Gibco | 14190-136 | 1929973 |
| Kasumi-3 cells | ATCC | CRL-2725 | 63990133 |
| | (Manassas, VA) | | |
| BD Cytofix buffer | BD Biosciences | 554655 | 7271598 |
| | (Franklin | | |
| | Lakes, NJ) | | |
| Dimethyl sulfoxide | Sigma (St. | D2650 | RNBG1808 |
| (DMSO) | Louis, MO) | | |
| Zoledronic acid | Sigma | SML0223 | 064M4703 |
| Monohydrate | | (10 mg) | |
| PE Anti Human TCR | Biolegend | 331308 | B253981 |
| Vγ9(clone B3) | | | |
| PE Anti Human TCR | Biolegend | 331408 | B212475 |
| Vδ2 (clone B6) | | | |
| PE Anti human | Biolegend | 372208 | B247485 |
| Granzyme B | | | |
| APC Anti Human | Biolegend | 331212 | B248353 |
| TCR γδ (clone B1) | | | |
| APC Anti human TCR | Biolegend | 331310 | B246678 |
| Vγ9(clone B3) | | | |
| Alexa 647 Anti | Biolegend | 310918 | B246313 |
| Human CD69 | | | |
| Alexa fluor Anti | Biolegend | 338814 | B256884 |
| Human CD44 | | | |
| PE Cy7 Anti Human | Biolegend | 372714 | B264577 |
| TIGIT (Clone VSTM3) | | | |
| FITC Anti Human | Biolegend | 329904 | B253784 |
| PD1(CD279) | | | |
| TCR Vδ1 monoclonal | Invitrogen | TCR2730 | TA259706 |
| antibody (TS8.2) | (Carlsbad, CA) | | |
| APC Anti Human IL-10 | Biolegend | 506807 | B248291 |
| FITC Anti-Human IL-6 | Biolegend | 501104 | B223492 |
| APC Anti-Human | Biolegend | 372204 | B248651 |
| Granzyme B | | | |
| Brilliant Violet 510 | Biolegend | 512330 | B236559 |
| Anti Human IL-17A | | | |
| Brilliant Violet | Biolegend | 502948 | B244147 |
| 785 Anti Human TNFα | | | |
| Brilliant Violet | Biolegend | 502538 | B238336 |
| 650 Anti Human IFNγ | | | |
| Brilliant Violet | Biolegend | 500338 | B230073 |
| 510 Anti Human IL-2 | | | |
| Brilliant Violet 711 | Biolegend | 308130 | B242465 |
| Anti Human Perforin | | | |
| Brilliant Violet | Biolegend | 345028 | B251351 |
| 650 Anti Human Tim-3 | | | |
| PE/Cy7 Anti Human | Biolegend | 369614 | B240088 |
| CD 152 (CTLA-4) | | | |
| APC/Cy7 Anti Human | Biolegend | 300518 | B255536 |
| CD4 | | | |
| Alexa Flour 488 Anti | Biolegend | 369326 | B261717 |
| Human CD223(Lag3) | | | |
| Alexa Flour 700 Anti | Biolegend | 344724 | B242233 |
| Human CD8 | | | |
| Biotin Anti Mouse | Biolegend | 302804 | |
| CD27 | | | |
| PE/Cy7 Anti Human | Biolegend | 304230 | B249604 |
| CD45RO | | | |
| FITC Anti Human | Biolegend | 304148 | B228669 |
| CD45RA | | | |
| APC/Cy7 Anti Human | Biolegend | 310914 | B261588 |
| CD69 | | | |
| APC/Cy7 Streptavidin | Biolegend | 405208 | B258702 |
| EasySep Human γδ T | Stem Cell | 19255 | 17J83331 |
| cell isolation kit | technologies | | 18E91569 |
| | (Vancouver, | | 18E91790 |
| | Canada) | | |
| CleanCap ™EGFP | TriLink (San | L-7201- | Supplied by |
| mRNA | Diego, CA) | 100 | Janssen |
| Human Trustain Fc | Biolegend | 422302 | B247182 |
| Aqua Live/Dead | Thermo Fisher | L34955 | 1910686 |
| | Scientific | | |
| Easy Sep buffer | Stem cell | 20144 | 18D89444 |
| | Technologies | | |
| Neon transfection | Thermo Fisher | MPK1096 | 2K11311 |
| 10 μL kit | Scientific | | |
| Recombinant Human | R&D systems | 301-R3 | Supplied by |
| IL-3 R alpha/CD123 | | | Janssen |
| Protein | | | |
| BD Cytofix/Cytoperm | BD Biosciences | 554722 | 7156885 |
| BD Perm/Wash | BD Biosciences | 554723 | 7180888 |
| Recombinant Human | R&D systems | 247-1LB- | TLM1117061 |
| IL-15 | | 025 | |

Cell Culture

Kasumi-3 cells were cultured in RPMI+20% FBS+lx penicillin-streptomycin. Cells were passaged every 3 days. Briefly, Kasumi-3 cells were collected from the flask and centrifuged at 1500 rpm for 5 minutes. Supernatant was discarded and the cells were seeded back in fresh media at a density of 5×10⁵ cells/mL.

Selective Expansion of Vγ9 Positive γδ T Cells from Whole PBMCs

On day 0, a vial of frozen PBMCs was thawed and added to 49 mL of warm complete RPMI (RPMI+10% FBS+1×Pen/Strep)) in a 50 mL falcon tube to dilute the freezing medium. The PBMCs were centrifuged at 1500 RPMI for 5 minutes. The PBMCs were washed once by re-suspending them in 35 ml of complete RPMI medium (RPMI+10% FBS+1×Pen/Strep). The cell pellet was re-suspended in the complete RPMI media and the cells were counted.

Alternatively, PBMCs were isolated from a whole blood sample by density gradient centrifugation and the cells were counted by using a hemocytometer. The cell count was adjusted to 1.0×10⁶ cells/1.0 mL in complete RPMI media.

Meanwhile, γδ T cell culture medium (RPMI-10%; RPMI supplemented with 10% FBS, 1×Pen/Strep) supplemented with recombinant human IL-2 (rhIL-2) to a final concentration of 1000 IU/mL; recombinant human rhIL-15 to a final concentration of 10 ng/mL; and Zoledronic acid (Zol) to a final concentration of 5 μM was prepared. The cell density was adjusted to 1×10⁶ cells/mL with the prepared γδ T cell culture media.

4×10⁶ cells were seeded in 4 mL of culture medium in a T-25 flask. On day 2, 4 mL of fresh γδ T cell culture medium supplemented with rhIL-2 to a concentration of 8001U/mL and rhIL-15 to a concentration of 20 ng/mL (the final concentration of rhIL-2 and rhIL-15 was 400 IU/mL and 10 ng/mL respectively) was added to the T-25 flask.

On day 5, the cells were centrifuged at 1500 rpm for 5 minutes. The supernatant was discarded and the cell pellet was re-suspended in 25 mL of complete RPMI medium supplemented with rhIL-2 to a final concentration of 100 IU/mL and rhIL-15 to a final concentration of 10 ng/mL. The cells were transferred into a T-75 flask, and the cell density was maintained at or around 1×10⁶ cells/mL throughout the expansion protocol.

On day 8 and 11, if the cell density was too high, cells were spun down at 1500 rpm for 5 minutes. Cells were split into a 1:2 ratio in 25 ml of complete RPMI medium supplemented with rhIL-2 to a final concentration of 100 IU/mL and rhIL-15 to a final concentration of 10 ng/mL.

Isolation and Enrichment of γδ T Cells

Isolation and enrichment of γδ T cells was performed using EasySep′ Human γδ T cell isolation kit (Stem cell Technologies; Vancouver, Canada) according to manufacturer instructions.

Isolation of γδ T Cells from Whole PBMCs,

Frozen PBMCs were thawed and added to 49 mL of warm complete RPMI media in a 50 mL falcon to dilute the freezing medium. The PBMCs were centrifuged at 1500 rpm for 5 minutes, and the PBMCs were washed once with complete RPMI media (RPMI+10% FBS+1×Pen/Strep). The cell pellet was re-suspended in 1 mL of Easy Sep buffer and the cells were counted by using a hemocytometer.

Isolation and Enrichment of γδ T Cells from Cultured PBMCs

Cells were harvested and centrifuged at 1500 rpm for 5 minutes. The cells were washed once by re-suspending them in plain RPMI (no FBS, or Pen/Strep) medium, and were centrifuged at 1500 rpm for 5 minutes. The cell pellet was re-suspended in 1 mL of Easy Sep buffer and the cells were counted by using a hemocytometer. The cell number was adjusted to 50×10⁶ cells in 1 mL of Easy Sep buffer in a 5 mL polystyrene round bottom tube.

50 μL of biotinylated cocktail was added to the re-suspended cells. The cells were mixed and incubated at room temperature for 10 minutes. Before the end of the incubation period, magnetic particles were vortexed for 30 seconds to get them evenly dispersed. After the incubation period, 50 μL of magnetic particles were added to 1 mL of the re-suspended cells. The cell suspension was mixed and incubated at room temperature for 5 minutes. After the incubation period, 1.5 mL of Easy Sep buffer was added to the tube containing the cells. The cells were re-suspended by gently mixing the cells up and down, and the tube was placed into the magnet and incubated for 5 minutes at room temperature. At the end of the incubation period, the media containing the enriched cell suspension was collected by inverting the magnet (containing the tube) in one continuous motion.

To the enriched cell suspension, 37 μl of vortexed magnetic particles were added, and the cell suspension was mixed and incubated at room temperature for 5 minutes. The tube containing the cell suspension was placed into the magnet and incubated at room temperature for 5 minutes. Enriched cells were collected by transferring the cell suspension into a new 15 mL tube. 10 mL of complete RPMI media was then added to the tube, and the cells were centrifuged at 1500 rpm for 5 minutes. The cells were washed one more time by adding 10 mL of complete RPMI medium, and then centrifuged at 1500 rpm for 5 minutes. The cell pellet was re-suspended in 1 mL of complete RPMI medium and the cells were counted by using a hemocytometer. The purity of the enriched cells was checked on a flow cytometer by staining the cells with TCR γδ, TCR αβ and TCR Vγ9 antibodies.

Phenotypic Profiling of γδ T Cells

Surface Phenotype Profiling

Fresh or cultured PBMCs were harvested, washed once with FACS buffer (PBS+2% FBS) and were counted using a hemocytometer. 2×10⁶ fresh PBMCs or 0.5×10⁶ Zol+rhIL-2+rhIL-15 or rhIL-2+rhIL-15 expanded cells were centrifuged at 1500 rpm for 15 minutes in a 96-well V-bottom plate. The supernatant was discarded and the cell pellet was re-suspended in 100 μL of PBS containing 0.5 μL of Live/Dead fixable violet dead cell stain, and the cells were incubated at room temperature for 20 minutes. The cells were centrifuged at 1500 rpm for 5 minutes, and the cell pellet was washed once with 200 μL of FACS buffer (PBS+2% FBS). The cell pellet was re-suspended in 100 μL of FACS buffer (PBS+2% FBS) containing 5 μL of Human Trustain Fc block, and the cells were incubated in the dark at 4° C. for 20 minutes. After incubation, the cells were centrifuged at 1500 rpm for 5 minutes.

Alternatively, 1×10⁶ cells were incubated in a 96-well V-bottom plate containing 100 μL of PBS containing both Live/Dead fixable violet dead cell stain (0.5 μL) and Human Trustain Fc block (5 μL) at 4° C. in dark. After incubation, the cells were centrifuged at 1500 rpm for 5 minutes, and the cells were washed once with 800 μL of FACS buffer (PBS+2% FBS). The cells were stained by incubating in 100 μL of FACS buffer containing an antibodies master mix at 4° C. for 30 minutes. After the incubation period, the cells were centrifuged at 1500 rpm for 5 minutes, and the cells were washed twice with 200 μL of FACS buffer and re-suspended in 100 μL of FACS buffer. Cells were acquired on the flow cytometer (Novocyte).

Intracellular Effector Molecule Profiling

One million fresh PBMCs or day 8 Zol activated PBMCs were incubated in 100 μL of PBS containing 0.5 μL LIVE/DEAD Fixable Violet Dead Cell Stain and 5 μL Fc block. The cells were incubated for 20 minutes at 4° C. in dark. After the incubation period, the cells were centrifuged at 1500 rpm for 5 minutes, and the cells were washed twice by re-suspending them in 200 μl of FACS buffer (PBS+2% FBS).

The cells were surface stained in 100 μL volume by incubating them with antibodies specific for Vγ9, Vδ2 for 30 minutes at 4° C. in dark. After the incubation period, 100 μL of FACS buffer (PBS+2% FBS) was added to the cells and the cells were centrifuged at 1500 rpm for 5 minutes. The supernatant was discarded, and the cells were washed twice by re-suspending the cell pellet in 200 μL of FACS buffer. The cells were centrifuged at 1500 rpm for 5 minutes, and the supernatant was discarded.

The cells were fixed by re-suspending the cell pellet in 100 μL BD Cytofix/Cytoperm buffer, and the cell suspension was incubated at 4° C. for 15 minutes in the dark. After the incubation period, the cells were centrifuged at 1500 rpm for 5 minutes. The supernatant was discarded, and the cells were washed by re-suspending the cell pellet in 200 μL of 1×BD Perm/Wash. The cells were centrifuged at 1500 rpm for 5 minutes. The supernatant was discarded, and the cell pellet was re-suspended in 100 μL of 1× Perm/Wash containing antibodies against intracellular antigens (Granzyme B, Perforin). The cell suspension was incubated at 4° C. for 30 minutes in the dark. After the incubation period, the cells were centrifuged at 1500 rpm for 5 minutes by adding 150 μL of 1×BD Perm/Wash. The cells were washed one more time by re-suspending the cell pellet in 200 μL of 1×BD Perm/Wash, and the cells were centrifuged at 1500 rpm for 5 minutes. The supernatant was discarded, and the cell pellet was re-suspended in 100 μL of FACS buffer (PBS+2% FBS). The cells were acquired on a Novocyte flow cytometer.

Detection of CD123 Expression on Kasumi-3 Cells

Fifty thousand Kasumi-3 cells were stained in 100 μL of FACS buffer (1×PBS+2% FBS). The cells were centrifuged at 1500 rpm for 5 minutes, and the supernatant was discarded. Anti-human CD123 antibody was added to the cells at a concentration of 2 μg/mL in FACS buffer (1×PBS+2% FBS) along with the respective isotype. An aliquot of the sample was left unstained. The cells were incubated at 4° C. for 30 minutes in the dark. Following incubation, the cells were centrifuged at 1500 rpm for 5 minutes and washed with FACS buffer (lx PBS+2% FBS) to remove any unbound antibodies. The washing step was repeated one more time (altogether, two washes were given), and the stained samples were fixed by re-suspending the stained cells in 100 μL of BD Cytofix for 15 minutes on ice. After the incubation period, the cells were washed once with FACS buffer and re-suspended in FACS buffer. Stained cells were acquired on a flow cytometer (BD FACS Calibur) followed by analysis by Flow Jo (version 10.3). Gating was done based on isotype controls.

Electroporation of mRNA into Activated T Cells

For Whole PBMCs

PBMCs cultured in complete RPMI medium containing Zol+rhIL-2+rhIL-15 for 8 days were harvested and centrifuged at 1500 rpm for 5 minutes. The cells were washed with plain RPMI medium (no FBS or Pen/Strep) by re-suspending the cell pellet in 35 mL of medium. The cells were centrifuged at 1500 rpm for 5 minutes, the cell pellet was re-suspended in 5 mL of complete RPMI medium, and the cells were counted by using a hemocytometer.

1×10⁶ cells were centrifuged in a 1.5 mL of Eppendorf tube at room temperature at 1400 rpm for 5 minutes, and the supernatant was discarded. The cell pellet was re-suspended in buffer T (2×10⁵ cells/9 μL of buffer T) from a Neon transfection kit (Thermo Fisher Scientific). 1 μL of GFP mRNA (1 μg/μL concentration) was added to the cell suspension, and the cell suspension was gently mixed by pipetting the cell suspension up and down for two times.

The Neon Tip (10 μL tip) was prepared by taking it from the Neon tip box with a Neon pipette and gently pressed up and down to remove any trapped air. 10 μL of cell suspension (9 μL of cell suspension+1 μL of mRNA) was slowly taken into the Neon tip. Meanwhile, the Neon tube was prepared by adding 3.5 mL of electrolyte buffer (buffer E). The Neon Tip containing the cell suspension was slowly placed into the Neon Tube containing the electrolyte buffer. The Neon tube (containing the Neon tip) was placed into the Neon docking station.

Electroporation was performed with the designated voltage, pulse width and number of pulses. Immediately after electroporation, the Neon tip (containing the cell suspension and mRNA) was removed from the Neon tube and the cells were added to a 48-well plate containing 0.5 mL of pre-warmed RPMI medium containing 10% FBS, without antibiotics. The plate was gently rocked to ensure the even distribution of the cells in the well, and the plate was incubated at 37° C. in a humidified CO₂ incubator. After 2 hours, 4 hours and 24 hours of incubation, 1/10 volume of the medium containing cells was taken and GFP fluorescence was determined on a Novocyte flow cytometer.

For Enriched γδ T Cells

Whole PBMCs cultured with Zol+rhIL-2+rhIL-15 for 14 days were harvested and centrifuged at 1500 rpm for 5 minutes. The cells were washed by re-suspending the cell pellet with 35 mL of plain RPMI medium. The cells were centrifuged at 1500 rpm for 5 minutes. γδ T cells were enriched via negative selection (as detailed above). The cells were washed once with complete RPMI medium (RPMI+10% FBS+1×Pen/Strep), and the cells were centrifuged at 1500 rpm for 5 minutes. The cell pellet was re-suspended gently with buffer T (2×10⁵ cells/9 μL of buffer T) from the Neon transfection kit (Thermo Fisher Scientific). 1 μL of GFP/CAR mRNA (GFP mRNA concentration CAR mRNA concentration 1.4 μg/μL) was added to the cell suspension. The cell suspension was gently mixed by pipetting the cell suspension up and down for two times.

The Neon Tip (10 μL tip) was prepared by taking it from the Neon tip box with a Neon pipette and gently pressed up and down to remove any trapped air. 10 μL of the cell suspension was slowly taken (9 μL of cell suspension+1 μL of mRNA) into the Neon tip. Meanwhile, the Neon tube was prepared by adding 3.5 mL of electrolyte buffer (buffer E). The Neon Tip containing the cell suspension was slowly placed into the Neon Tube containing electrolyte buffer, and the Neon tube (containing the Neon tip) was placed into the Neon docking station.

Electroporation was performed at 1400V, 20 ms pulse width and 1 pulse.

Immediately after electroporation, the Neon tip (containing cell suspension and mRNA) was removed from the Neon tube and the cells were added to a 48-well plate containing 0.5 mL of pre-warmed RPMI medium containing 10% FBS, without antibiotics. The plate was gently rocked to ensure the even distribution of the cells in the well. The plate was incubated at 37° C. in a humidified CO₂ incubator. As a no-electroporation control, 1 μL of mRNA (GFP or CAR) was added to 9 μL of Buffer T that contains 2×10⁵ enriched γδ T cells. Cells were added to a 48-well plate containing 0.5 mL of pre-warmed RPMI medium containing 10% FBS, but no antibiotics. The plate was gently rocked to ensure the even distribution of the cells in the well. The plate was incubated at 37° C. in a humidified CO₂ incubator. Cell viability (by measuring cells negative for Live/Dead staining) and the transfection efficiency (by measuring the frequency of GFP⁺ cells) were determined by taking 1/10 volume (50 μL) of the culture media containing the cells after 2 hours, 20 hours and 40 hours of culture period. Cells were rested for 40 hours after electroporation and used as effector cells for the cytotoxicity experiment.

CAR Transfected γδ T Cells Mediated Cytotoxicity Assay

Labeling Target Cells (Kasumi-3) with CFSE

Kasumi-3 cells were harvested and washed once with plain RPMI medium. The cells were counted and the density of the cells was adjusted to 1×10⁶ cells/mL. The cells were re-suspended in 1 mL of 0.5 μM CFSE in 1×PBS and incubated for 8 minutes at room temperature (RT) with occasional mixing. One mL of FBS was added to stop the labelling reaction. The cells were washed twice in complete RPMI media (RPMI+10% FBS+1×Pen/Strep). The cells were counted using a hemocytometer, and the cell density was adjusted in 100 μL volume according to ET ratio.

Preparing Effector (CAR-Gd T Cells) Cells

γδ T cells were enriched from total PBMCs that were cultured in complete RPMI medium (RPMI+10% FBS+1×Pen/Strep) containing Zol+rhIL-2+rhIL-15 for 14 days. γδ T cell purity was assessed post enrichment by staining the cells with TCR γδ, TCR αβ, TCR Vγ9 monoclonal antibodies by flow cytometry.

Chimeric Antigen Receptor (CAR) mRNA electroporation was carried out on enriched γδ T cells with Neon electroporation system at 1400V, 20 ms pulse width, 1 pulse. After electroporation, γδ cells were rested for 24 and 40 hours. After resting period, γδ cells (effector cells) were used for the cytotoxicity experiment.

Effector to Target Ratio

10,000 target cells were added to each well (in a 100 μL volume of complete RPMI medium). 10,000 CAR/GFP transfected enriched γδ T cells in 100 μL volume of complete RPMI medium were added, and the cells were incubated at 37° C. and 5% CO₂ for the indicated time points

Acquisition and Analysis

For surface phenotype profiling experiments, cells were initially gated on FSC-H vs SSC-H on total cells. Live cells were gated in from total cells. Doublets were eliminated from live cells by gating on FSC-A Vs FSC-H parameters. From here, surface profiling was done by gating on Vγ9⁺ cells. For cytotoxicity assays, stained cells were acquired on a BD FACS Calibur/Novocyte instrument with Cell Quest Pro (version 6.0)/novo express software respectively. Data was transferred via general folder to a desktop with Flow Jo (version 10.3). CFSE positive cells were first gated on to identify the target cells. Within the CFSE positive cells, dead cells were identified as 7-AAD⁺ FSC^{low} cells. Gates were set based on the CFSE unstained and 7-AAD unstained cells. To calculate the CAR transduced γδ T cell specific lysis, spontaneous target cell lysis values were subtracted from cell lysis values obtained from wells containing GFP or I3RB135_LH or I3RB135_HL mRNA electroporated γδ T cells.

Results

### Example 1: Distribution of γδ T Cell Subsets in Whole Fresh PBMCs

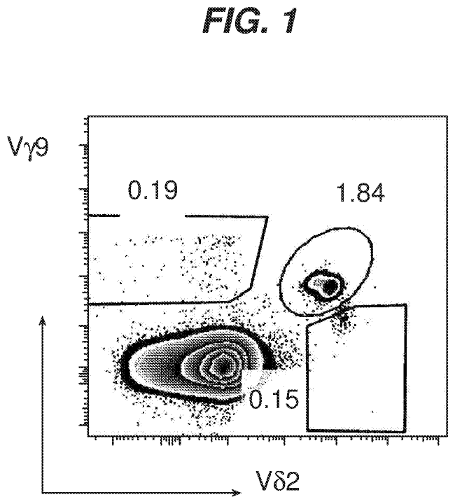

To identify frequency of TCR Vγ9⁺Vδ2⁺ cells among total PBMCs, cells were stained with anti-TCR Vγ9 and TCR Vδ2 antibody. Flow cytometry analysis showed that Vγ9 positive cells predominantly pair with Vδ2 and the frequency of these cells is around 1.8% among total PBMCs (FIG. 1).

### Example 2: Stimulation and Expansion of Vγ9⁺ Cells from Total PBMCs

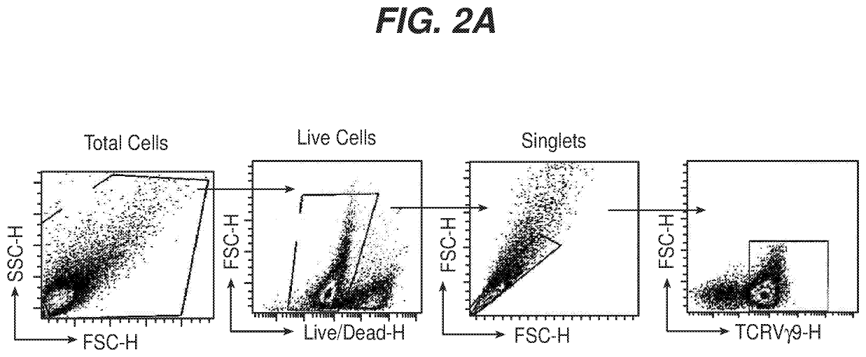

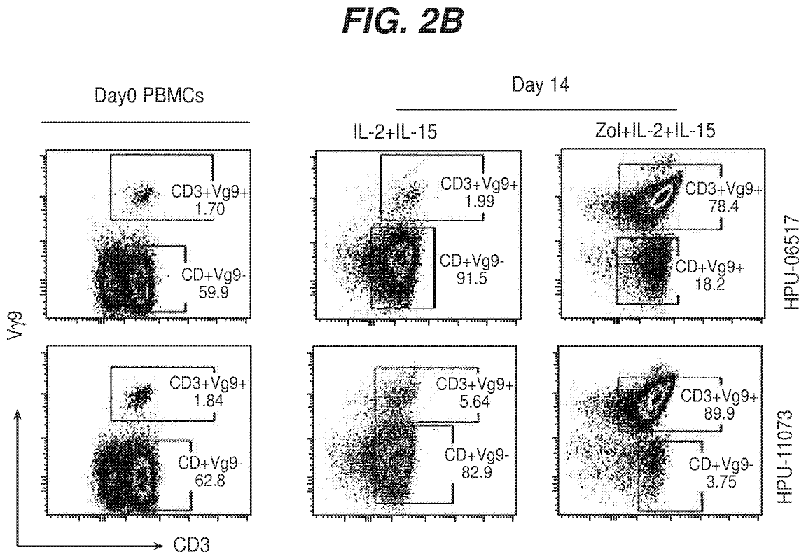

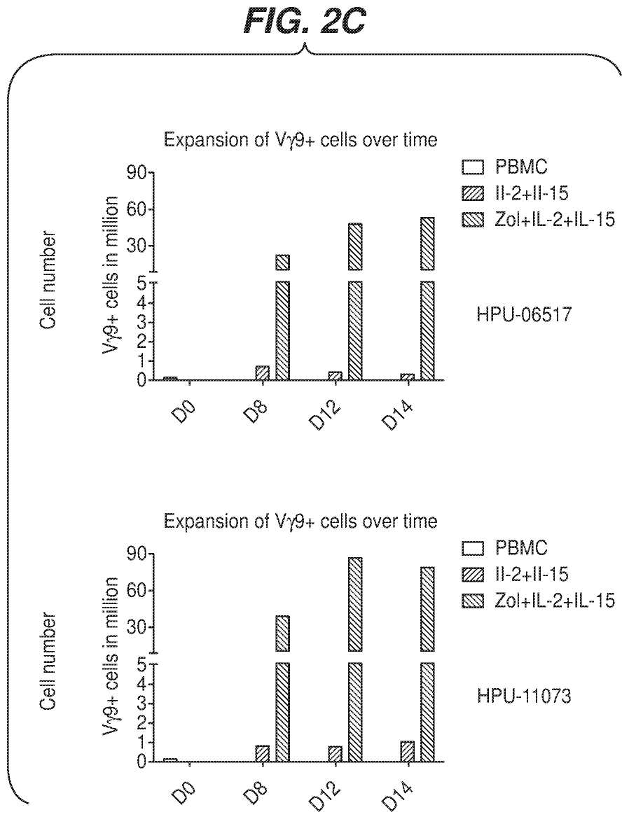

To selectively expand Vγ9⁺γδ T cells, whole PBMCs were cultured in complete RPMI medium (RPMI+10% FBS+1×Pen/Strep) enriched with rhIL-2+rhIL-15+Zol. As a control, PBMCs were cultured in complete RPMI medium (RPMI+10% FBS+1×Pen/Strep) containing rhIL-2+rhIL-15 alone. Frequency of Vγ9⁺ cells among total PBMCs was determined on day 0 (among fresh PBMCs), day 8, and day 14 of the culture period. To enumerate the frequency of Vγ9⁺ cells among total PBMCs, total live PBMCs were initially gated, doublets were excluded, and Vγ9⁺ cells were gated (FIG. 2A). Compared to the frequency of Vγ9⁺ cells among total PBMCs (from two different donors) on day 0, a substantial and selective expansion of Vγ9⁺ cells was observed only in the presence of Zol on day 8, 12, and 14 of the culture period (FIGS. 2B and 2C).

### Example 3: Phenotype of Resting and Activated Vγ9⁺ γδ T Cells

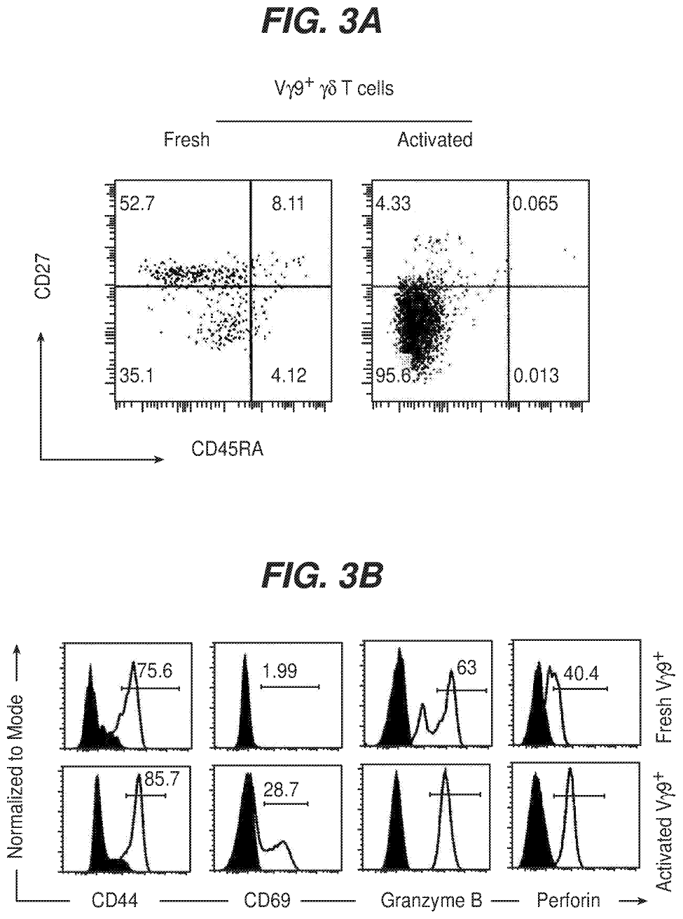

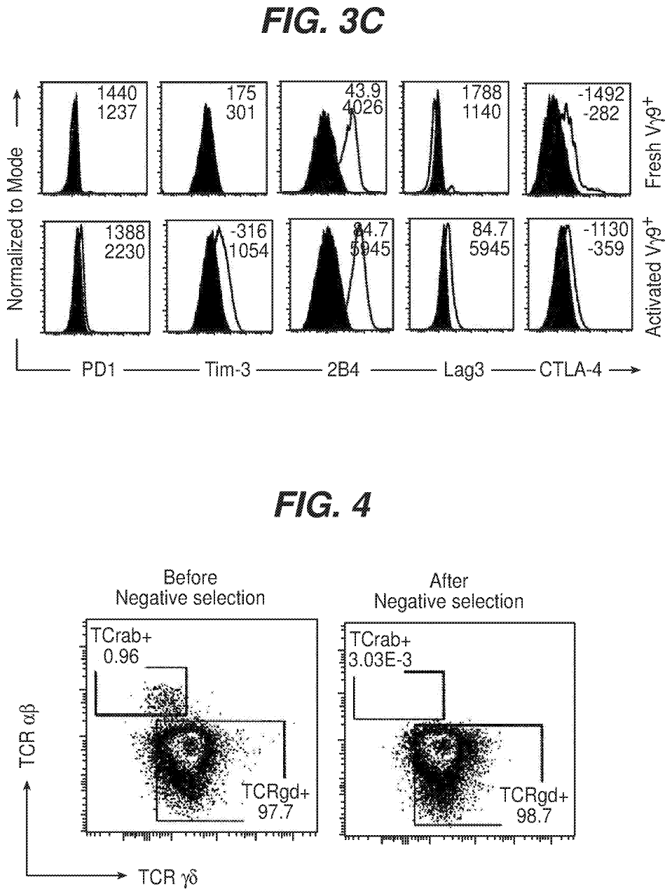

To understand the phenotypic differences between resting and activated Vγ9⁺γδ T cells, fresh PBMCs and day 8 Zol activated PBMCs were stained with anti-TCRγδ, Vγ9 antibody to initially identify Vγ9⁺γδ T cells. Upon profiling Vγ9⁺γδ T cells with differentiation markers (CD45RA, CD27), resting Vγ9⁺γδ T cells showed predominantly central memory (CD27⁺CD45RA⁻) and effector memory (CD27⁻CD45RA⁻) phenotypes. While Zol activated Vγ9⁺γδ T cells showed exclusively effector memory (CD27⁻ CD45RA⁻) phenotype (FIG. 3A). In line with this, resting Vγ9⁺γδ T cells (from fresh PBMCs) showed an activated phenotype (CD44 surface expression), accompanied by a prominent intracellular Granzyme B and Perforin expression (FIG. 3B). Similarly, Zol activated Vγ9⁺γδ T cells showed a prominent expression of CD44 on their surface along with intracellular Granzyme B and Perforin expression (FIG. 3B). Interestingly, resting Vγ9⁺γδ T cells did not show any surface expression of CD69, an early activation marker. While, a fraction (28%) of Zol activated Vγ9⁺γδ T cells did show the presence of CD69 on their surface (FIG. 3B).

Upon staining Vγ9⁺γδ T cells with various inhibitory receptors, resting Vγ9⁺γδ T cells showed no surface expression of PD1, Tim-3 and CTLA-4. On the other hand, activated Vγ9⁺γδ T cells showed a prominent up regulation of Tim-3 on the cell surface. However, PD1 and CTLA-4 expression on activated Vγ9⁺ cells seemed unchanged compared to resting Vγ9⁺γδ T cells. Interestingly, a large fraction of resting Vγ9⁺γδ T cells express 2B4 on their surface and virtually all Vγ9⁺γδ T cells expressed 2B4 on their surface upon their activation with Zol (FIG. 3C).

### Example 4: Negative Selection to Deplete αβ T Cells

γδ T cells were enriched from day 14 Zol+rhIL-2+rhIL-15 culture via negative selection using EasySep Human γδ T cell isolation kit. γδ T cell enrichment eliminated residual TCRαβ T cell contamination (FIG. 4). CAR mRNA transfection into γδ T cells was performed with the optimized electroporation parameters (1400 V, 20 ms pulse width and 1 pulse).

### Example 5: Design and Construction of CAR-T

The CAR-T construct consisted of an antigen specific single chain Fv (scFv) moiety anchored on the membrane with a flexible human CD8 hinge sequence. A single pass transmembrane domain of human CD8 followed by a co-stimulation moiety (4-1BB) and a signaling domain (CD3ζ) was also part of the construct (FIG. 7).

### Example 6: Cytotoxicity of CAR Transduced γδ T Cells

In order to analyze the CAR mediated γδ T cell cytotoxicity, CFSE labelled target (Kasumi-3) cells were co-cultured with CAR transfected γδ T cells (effectors) from day 14 of culture at an effector to target (ET) ratio of 1:1 for 24 hours. As a control, CFSE labelled target (Kasumi-3) cells were also cultured with GFP transfected γδ T cells at an effector to target (ET) ratio of 1:1. After electroporation, cells were rested for a period of 40 hours.

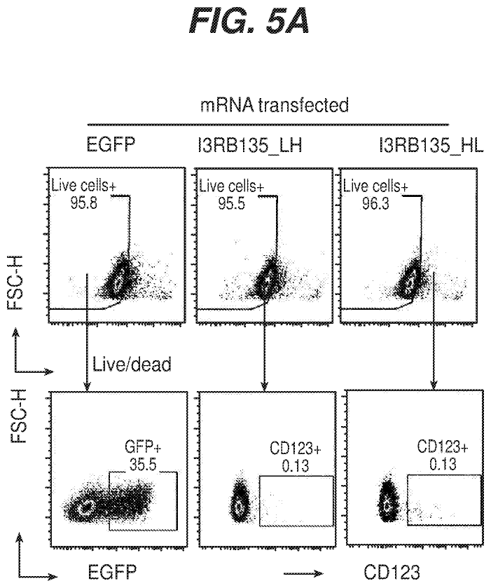

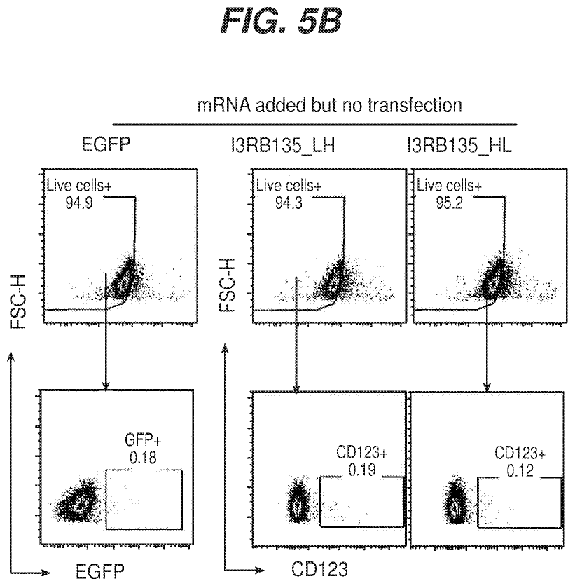

Cell viability and the transfection efficiency of these cells were captured during their resting period by measuring their ability to exclude LIVE/DEAD stain and EGFP expression respectively at 2, 20 and 40 hours post transfection (FIGS. 5A and 5B). Cell viability was close to 96% at 2 hours post electroporation and went down to around 60% by 40 hours post electroporation. Transfection efficiency (% of EGFP⁺ cells) was at 35% by 2 hours post electroporation and was marginally increased to 42% by 20 hours. As a transfection control, γδ T cells were treated similarly by adding CAR/GFP mRNA and left them un-transfected (FIG. 5A, right).

CD123 expression on target (Kasumi-3) cells was analyzed by using a commercially available anti-CD123 antibody. CAR surface expression was measured by probing the cells with 1 μg of CD123 biotinylated purified protein. Streptavidin PE/Cy7 was used to label the biotinylated protein. PE/Cy7 signal on CAR transfected γδ T cells was not observed, which could potentially be due to the fact that the biotinylation of CD123 purified protein failed or for other technical reasons.

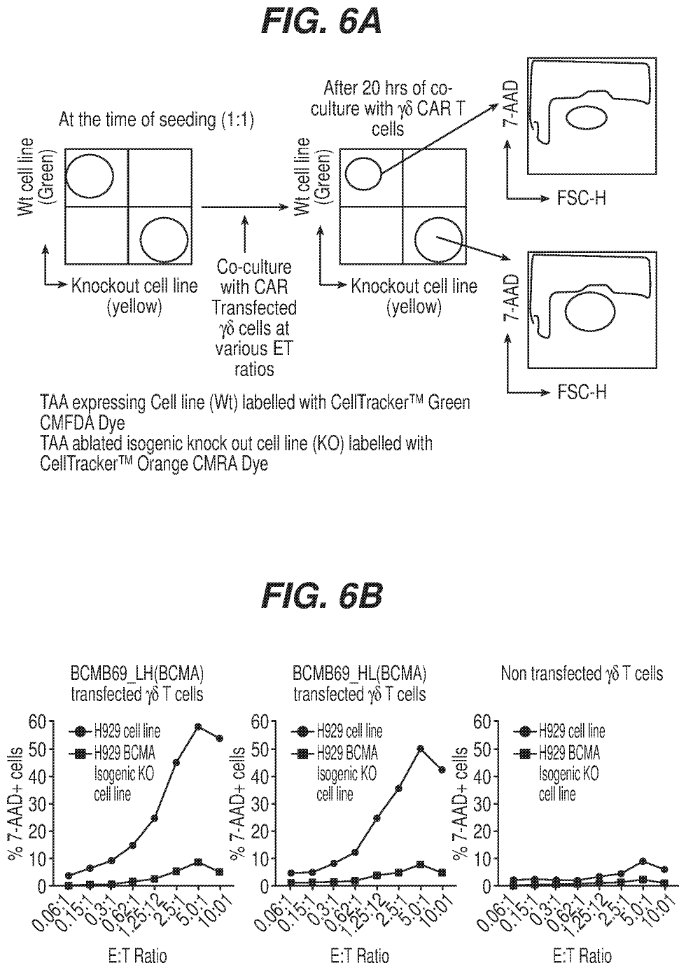

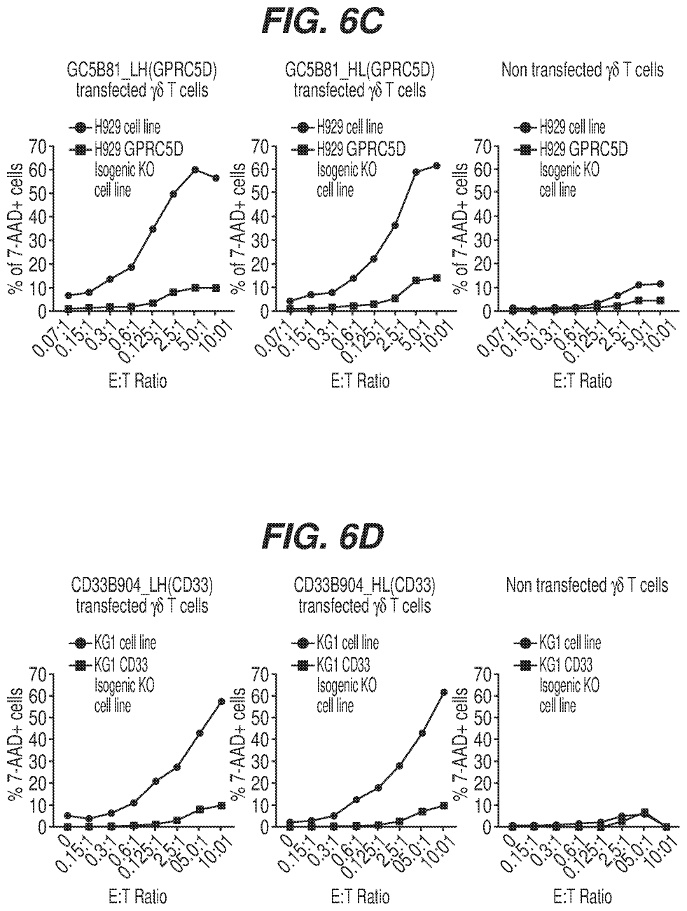

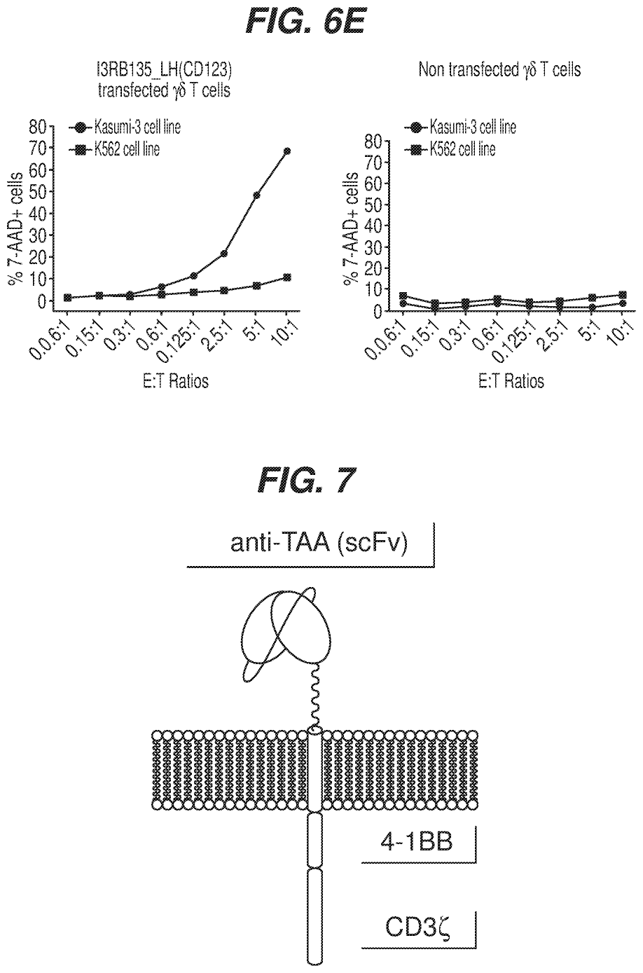

Target antigen expressing cells (such as Kasumi-3 for CD123, H929 cells for GPRC5D, KG1 cells for CD33) and target knockout cells or non-target expressing cells (such as K562 for CD123) were labelled with CFSE to identify them as CFSE⁺ during flow cytometry analysis. Post co-culture period, 7-AAD was added to analyze the percentage of 7-AAD⁺ CFSE⁺ cells as a measure of cytotoxicity. Basal cytotoxicity observed with un-transfected γδ T cells was subtracted to obtain specific cytotoxicity mediated by CAR expression on γδ T cells. Maximum cytotoxicity observed with γδ T cells transfected with mRNA constructs were ranging from 60% to −70%, (FIG. 6B-E).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of expanding and isolating γδ T cells from human peripheral blood mononuclear cells (PBMCs), the method comprising:
a. obtaining human PBMCs;
b. culturing the human PBMCs in a culture media comprising zoledronic acid, interleukin-2 (IL-2), and interleukin-15 (IL-15) to expand the γδ T cell; and
c. isolating the γδ T cells.

2. The method of claim 1, wherein the concentration of the zoledronic acid is about 1 μM to about 20 μM.

3. The method of claim 2, wherein the concentration of the zoledronic acid is about 5 μM.

4. The method of claim 1, wherein the concentration of the IL-2 is about 50 IU/mL to about 5000 IU/mL.

5. The method of claim 4, wherein the concentration of IL-2 is about 100 IU/mL to about 1000 IU/mL.

6. The method of claim 1, wherein the IL-2 is recombinant human IL-2 (rhIL-2).

7. The method of claim 1, wherein the concentration of IL-15 is about 1 ng/mL to about 100 ng/mL.

8. The method of claim 7, wherein the concentration of IL-15 is about 10 ng/mL.

9. The method of claim 1, wherein the IL-15 is recombinant human IL-15 (rhIL-15).

10. The method of claim 1, wherein the γδ T cell is a Vγ9Vδ2 T cell.

11. The method of claim 1, wherein the γδ T cells are isolated by flow cytometry, magnetic separation, and negative selection.

12. An isolated γδ T cell produced by the method of claim 11.

13. A method of generating a chimeric antigen receptor (CAR)-γδ T cell, the method comprising:
a. obtaining an isolated γδ T cell of claim 12;
b. contacting the γδ T cell with a nucleic acid encoding a chimeric antigen receptor (CAR), the CAR comprising:
i. an extracellular domain;
ii. a transmembrane domain; and
iii. an intracellular signaling domain,
wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

14. The method of claim 13, wherein the CAR comprises:
i. an extracellular domain comprising an antigen binding domain and/or an antigen binding fragment;
ii. a transmembrane domain comprising a CD8α transmembrane domain;
iii. an intracellular signaling domain comprising a CD3ζ or 4-1BB intracellular domain;
iv. a signal peptide comprising a CD8α signal peptide; and
v. a hinge region comprising a CD8α hinge region.

15. The method of claim 14, wherein the CAR comprises:
i. the transmembrane domain having an amino acid sequence at least 90% identical to SEQ ID NO:1;
ii. the intracellular domain having an amino acid sequence at least 90% identical to SEQ ID NO:2 or SEQ ID NO:3;
iii. the signal peptide having an amino acid sequence at least 90% identical to SEQ ID NO:4; and
iv. the hinge region having an amino acid sequence at least 90% identical to SEQ ID NO:5.

16. The method of claim 14, wherein the extracellular domain comprises an antigen binding domain and/or an antigen binding fragment that specifically binds a tumor antigen.

17. The method of claim 13, wherein the CAR comprises an amino acid sequence selected from the group consisting of SEQ ID NOs:22-29.

18. A CAR-γδ T cell produced by the method of claim 13.

19. A pharmaceutical composition comprising the CAR-γδ T cell of claim 18 and a pharmaceutically acceptable carrier.

20. A method of treating or preventing a disease or condition in a subject in need thereof, the method comprising administering a therapeutically effective amount of the pharmaceutical composition of claim 19.

21. The method of claim 20, wherein the disease or condition is cancer.

22. The method of claim 21, wherein the cancer is selected from a solid cancer or a liquid cancer.

23. The method of claim 22, wherein the cancer is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

24. The method of claim 20, wherein the disease or condition is an autoimmune disease.

25. The method of claim 24, wherein the autoimmune disease is selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere;s disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

26. A method of producing a pharmaceutical composition comprising a CAR-γδ T cell, wherein the methods comprises combining the CAR-γδ T cell of claim 18 with a pharmaceutically acceptable carrier to obtain the pharmaceutical composition.

27. The method of claim 15, wherein the extracellular domain comprises an antigen binding domain and/or an antigen binding fragment that specifically binds a tumor antigen.

28. The method of claim 14, wherein the CAR comprises an amino acid sequence selected from the group consisting of SEQ ID NOs:22-29.

29. The method of claim 15, wherein the CAR comprises an amino acid sequence selected from the group consisting of SEQ ID NOs:22-29.

30. The method of claim 16, wherein the CAR comprises an amino acid sequence selected from the group consisting of SEQ ID NOs:22-29.

31. A CAR-γδ T cell produced by the method of claim 13.
